(19) United States
(12) Patent Application Publication
(10) Pub. No.: US 2020/0078309 A1
(43) Pub. Date: Mar. 12, 2020

(54) Stable tablet compositions of Sacubitril: Valsartan

(71) Applicant: Hetero Labs Limited, Hyderabad (IN)

(72) Inventors: Parthasarathi Reddy BANDI, Hyderabad (IN); Khadgapathi PODILE, Hyderabad (IN); Sunil Deviprasad TIWARI, Hyderabad (IN); Naresh ANAPARTY, Hyderabad (IN)

(73) Assignee: Hetero Labs Limited, Hyderabad (IN)

(21) Appl. No.: 16/567,926

(22) Filed: Sep. 11, 2019

(30) Foreign Application Priority Data
Sep. 12, 2018 (IN) 201841034305

(51) Int. Cl. A61K 9/28; A61K 9/20; A61K 31/216; A61K 31/41

(52) U.S. Cl. CPC A61K 9/2866; A61K 9/2013; A61K 9/2027; A61K 31/41; A61K 9/2054; A61K 31/216; A61K 9/2009

(57) ABSTRACT

The present invention relates to pharmaceutical compositions of sacubitril; valsartan sodium and a process for the preparation thereof. Particularly, the present invention relates to stable non-aqueous based tablet composition of sacubitril; valsartan sodium and a process for the preparation thereof.

### FIELD OF THE INVENTION

The present invention relates to pharmaceutical compositions of amorphous sacubitril; valsartan sodium and process for the preparation thereof.

### BACKGROUND OF THE INVENTION

Sacubitril is a prodrug form of active metabolite Sacubitrilat. It belongs to class of drugs called neprilysin inhibitors that work by inhibiting neutral endopeptidase. Chemically Sacubitril is 4-{[(2S,4R)-1-(4-Biphenylyl)-5-ethoxy-4-methyl-5-oxo-2-pentanyl]amino}-4-oxobutanoic acid, having the following structural formula:

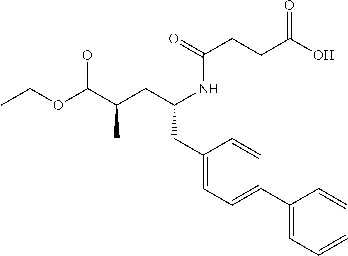 [SMILES: C=C/C=C(C=C)/C=C/C=C(\C=C)C[C@H](C[C@@H](C)C(O)OCC)NC(=O)CCC(=O)O]

Valsartan is a nonpeptide, orally active, and specific angiotensin II receptor blocker acting on the AT1 receptor subtype. Valsartan is described chemically as N-(1-oxopentyl)-N-[[2′-(1H-tetrazol-5-yl) [1,1′-biphenyl]-4-yl]methyl]-L-valine, having the following structural formula:

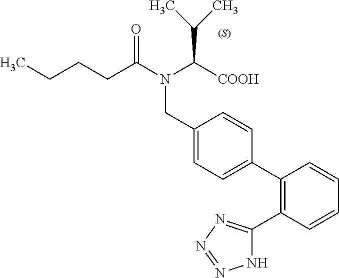

Sacubitril; Valsartan tablets are marketed in US under the brand name Entresto® by Novartis.

U.S. Pat. Nos. 5,217,996 and 5,399,578 disclose sacubitril and valsartan respectively.

U.S. Pat. No. 8,877,938 disclose crystalline valsartan; sacubitril trisodium hemipentahydrate.

U.S. Patent Publication 2010/0267786 disclose tablet composition comprising sacubitril; valsartan, prepared by roller compaction process.

PCT publication No. WO2017/037591 A1 disclose process for preparing amorphous Sacubitril/Valsartan sodium.

PCT publication No. WO2016/125123A1 disclose amorphous sacubitril/valsartan and its process for the preparation.

PCT publication No. WO2017/085573 A1 disclose amorphous sacubitril-valsartan complex. Also disclose amorphous solid dispersion of sacubitril-valsartan complex and its process for the preparation.

Despite different approaches disclosed in the art, there is a need to develop stable tablet compositions of sacubitril; valsartan sodium. Amorphous sacubitril-valsartan is deliquescent and prone to degradation and polymorphic conversion. The intention of the present invention was to provide tablet compositions having content uniformity and desired stability with low impurity profile. Inventors of the present invention have developed stable compositions of sacubitril; valsartan sodium using non-aqueous granulation.

### SUMMARY OF THE INVENTION

The present invention relates to process for preparing tablet composition comprising amorphous sacubitril; valsartan sodium and one or more pharmaceutically acceptable excipients.

One embodiment of the present invention relates to process for preparing tablet composition comprising non-aqueous granulation of amorphous sacubitril; valsartan sodium and one or more pharmaceutically acceptable excipients.

Another embodiment of the present invention relates to a process for the preparation of a tablet comprising non-aqueous granulation of (a) amorphous sacubitril; valsartan sodium comprising amorphous sacubitril; valsartan sodium and colloidal silicon dioxide, and (b) one or more pharmaceutically acceptable excipients.

Another embodiment of the present invention relates to a process for the preparation of a tablet comprising non-aqueous granulation of (a) amorphous sacubitril; valsartan sodium, and (b) one or more pharmaceutically acceptable excipients, wherein the tablet comprises (2R, 4S)-5-([1,1′-biphenyl]-4-yl)-4-(carboxypropanamido)-2-methyl pentanoic acid impurity in an amount of less than 0.2% by weight.

Another embodiment of the present invention relates to a process for the preparation of a tablet comprising amorphous sacubitril; valsartan sodium comprises: (a) sifting and blending amorphous sacubitril; valsartan sodium and one or more pharmaceutically acceptable excipients, (b) preparing granulating solution by dissolving binder in at least one non-aqueous solvent, (c) granulating the blend of step (a) using binder solution of step (b), followed by drying and milling to get the desired granules, (d) lubricating the granules of step (c) and compressing into tablets, (e) preparing film coating dispersion by dissolving a film coating polymer in at least one non-aqueous solvent, and finally, (f) coating the tablets of step (d) using dispersion of step (e), to get the final film coated tablets, wherein the process is carried out at a temperature of 25° C. and relative humidity of less than 50%.

Another embodiment of the present invention relates to process for preparing tablet composition comprising amorphous sacubitril; valsartan sodium and one or more pharmaceutically acceptable excipients preferably diluents in the range of 1% to 20%, binding agents in the range of 1% to 20%, disintegrants in the range of 0.1% to 10%, glidants in the range of 0.1% to 2% and lubricants in the range of 0.1% to 2%.

Another embodiment of the present invention relates to process for preparing tablet composition comprising amorphous sacubitril; valsartan sodium and one or more pharmaceutically acceptable excipients preferably about 1% to 20% of microcrystalline cellulose, about 1% to 20% of low-substituted hydroxypropylcellulose, about 1% to 10% povidone, about 0.1% to 10% of crospovidone, about 0.1% to 2% of colloidal silicon dioxide and about 0.1% to 2% of magnesium stearate.

Tablet composition of sacubitril; valsartan sodium according to the present invention is processed at a temperature of 25° C. and relative humidity of less than 50%, wherein the film coated tablet has (2R, 4S)-5-([1,1′-biphenyl]-4-yl)-4-(carboxypropanamido)-2-methyl pentanoic acid impurity in an amount of less than 0.2% by weight.

In yet another embodiment of the present invention relates to use of present composition to reduce the risk of cardiovascular death and hospitalization for heart failure in patients with chronic heart failure (NYHA Class II-IV) and reduced ejection fraction.

### DETAILED DESCRIPTION OF THE INVENTION

The present invention relates to process for preparing tablet composition comprising amorphous sacubitril; valsartan sodium and one or more pharmaceutically acceptable excipients.

Amorphous sacubitril-valsartan is deliquescent and prone to degradation and polymorphic conversion. The intention was to provide tablet compositions having content uniformity and desired stability with low impurity profile. Inventors of the present invention have developed stable compositions of sacubitril; valsartan sodium using non-aqueous granulation. The tablet compositions of the present invention have improved content uniformity and desired stability with low impurity profile. Inventors of the present invention found that the content uniformity and desired stability with low impurity profile was achieved when non-aqueous granulation was used in the process for preparing tablet compositions of sacubitril; valsartan sodium.

The term “pharmaceutically acceptable” as used herein means that which is useful in preparing a pharmaceutical composition that is generally safe and non-toxic.

The term “excipients” as used herein means a component of a pharmaceutical product that is not an active ingredient such as, for example, fillers, diluents, carriers and the like. The excipients that are useful in preparing a pharmaceutical composition are generally safe and non-toxic.

By the term “composition” as used herein refers to a solid dosage form suitable for oral administration, such as a tablet, capsule, spheroids, mini-tablets, pellets, granules, pills and the like; preferably, oral tablets.

The term “active ingredient” or “active agent” used interchangeably, is defined to mean active drug (e.g. sacubitril; valsartan) or its salt, that induce a desired pharmacological or physiological effect. The active ingredient of the present invention comprising amorphous sacubitril; valsartan sodium. Particularly, dissolving amorphous sacubitril; valsartan sodium and dispersing colloidal silicon dioxide in a non-aqueous solvent further distillation to form amorphous sacubitril; valsartan sodium on colloidal silicon dioxide; wherein the ratio of sacubitril; valsartan sodium to colloidal silicon dioxide is in the range of 1:0.5 to 1:1. Colloidal silicon dioxide was added to amorphous sacubitril; valsartan sodium to increase the stability and to reduce the hygroscopicity of amorphous sacubitril; valsartan sodium.

Excipients of the present invention comprise diluents, binders, disintegrants, glidants, lubricants and combinations thereof.

Diluents according to the present invention include but are not limited to microcrystalline cellulose, lactose, mannitol, dibasic calcium phosphate, tribasic calcium phosphate, calcium silicate, calcium carbonate, calcium sulfate, magnesium carbonate, magnesium oxide, talc, sugar, starches, sorbitol, inorganic salts, cellulose derivatives, calcium sulfate, xylitol, lactitol, kaolin, sucrose, sorbitol, dextrates, dextrin, maltodextrin, dextrose and the like, and combinations thereof.

Binders according to the present invention include but are not limited to polyvinyl pyrrolidone, hydroxypropyl cellulose, copovidone, hydroxypropyl methylcellulose, pregelatinized starch, powdered acacia, gelatin, guar gum, carbomers and the like, and combinations thereof.

Disintegrants according to the present invention include but are not limited to crospovidone, croscarmellose sodium, polacrilin potassium, sodium starch glycolate, carboxymethyl cellulose calcium, starches such as corn starch, potato starch, pre-gelatinized and modified starches, microcrystalline cellulose and the like, and combinations thereof.

Glidants according to the present invention include but are not limited to colloidal silicon dioxide (SYLOID® 244 FP, AEROSIL®, AEROPERL® or etc.), magnesium silicate, magnesium trisilicate, talc, and other forms of silicon dioxide, such as aggregated silicates and hydrated silica and the like, and combinations thereof.

Lubricants according to the present invention include but are not limited to magnesium stearate, aluminium stearate, sucrose stearate, stearic acid, talc, fumaric acid, palmitic acid, sodium stearyl fumarate, glyceryl monostearate, carnauba wax, hydrogenated vegetable oils, mineral oil, polyethylene glycols and the like and combinations thereof.

One embodiment of the present invention relates to process for preparing tablet composition of sacubitril-valsartan sodium comprising non-aqueous granulation of amorphous sacubitril-valsartan sodium and at least one pharmaceutically acceptable excipient.

In one aspect of an embodiment disclosed herein, (2R, 4S)-5-([1,1′-biphenyl]-4-yl)-4-(carboxypropanamido)-2-methyl pentanoic acid is one of the impurities observed during manufacturing process of sacubitril; valsartan sodium tablets.

In one aspect of an embodiment disclosed herein, tablet composition of amorphous sacubitril; valsartan comprises (2R, 4S)-5-([1,1′-biphenyl]-4-yl)-4-(Carboxypropanamido)-2-methyl pentanoic acid impurity in an amount of less than 0.2% by weight.

In yet another aspect of an embodiment disclosed herein, the tablet is coated with non-aqueous film coating.

Another embodiment of the present invention relates to process for preparing tablet composition of sacubitril-valsartan sodium comprising non-aqueous granulation of amorphous sacubitril-valsartan sodium and at least one pharmaceutically acceptable excipient, wherein amorphous sacubitril—valsartan sodium comprises colloidal silicon dioxide in a ratio of 1:0.5 to 1:1.

Non-aqueous solvents according to the present invention include but are not limited to isopropyl alcohol, dichloro methane, methanol, ethanol, ethyl acetate, ethyl lactate, acetone, methylenechloride, 1,1,1-trichloroethane, chloroform and the like and combinations thereof. Non-aqueous solvents can be useful for the preparation granulating solution and as a film coating solution.

In one aspect of an embodiment disclosed herein, tablet composition of amorphous sacubitril; valsartan sodium is processed at a temperature of 25° C. and relative humidity of less than 50%.

In one aspect of an embodiment disclosed herein, the film coated tablet of amorphous sacubitril; valsartan sodium is preferably, processed at a temperature of 25° C. and relative humidity of less than 40%.

Another embodiment of the present invention relates to a process for the preparation of a tablet comprising amorphous sacubitril; valsartan sodium comprises: (a) sifting and blending amorphous sacubitril; valsartan sodium and one or more pharmaceutically acceptable excipients, (b) preparing granulating solution by dissolving binder in at least one non-aqueous solvent, (c) granulating the blend of step (a) using binder solution of step (b), followed by drying and milling to get the desired granules, (d) lubricating the granules of step (c) and compressing into tablets, (e) preparing film coating dispersion by dissolving a film coating polymer in at least one non-aqueous solvent, and finally, (f) coating the tablets of step (d) using dispersion of step (e), to get the final film coated tablets wherein the process is carried out at a temperature of 25° C. and relative humidity of less than 50%.

Another embodiment of the present invention relates to process for preparing tablet composition comprising amorphous sacubitril; valsartan sodium and one or more pharmaceutically acceptable excipients preferably diluents in the range of 1% to 20%, binding agents in the range of 1% to 20%, disintegrants in the range of 0.1% to 10%, glidants in the range of 0.1% to 2% and lubricants in the range of 0.1% to 2%.

Another embodiment of the present invention relates to process for preparing tablet composition comprising amorphous sacubitril; valsartan sodium and one or more pharmaceutically acceptable excipients preferably about 1% to 20% of microcrystalline cellulose, about 1% to 20% of low-substituted hydroxypropylcellulose, about 1% to 10% povidone, about 0.1% to 10% of crospovidone, about 0.1% to 2% of colloidal silicon dioxide and about 0.1% to 2% of magnesium stearate.

Advantages of wet granulation include improvement of the cohesiveness, compressibility of powders, a good distribution and uniform content, reduction of a great deal of dust and airborne contamination, and prevention of segregation of components.

Film coated tablet of sacubitril; valsartan sodium according to the present invention is processed at a temperature of 25° C. and relative humidity of less than 50%, wherein the film coated tablet has a (2R, 4S)-5-([1,1′-biphenyl]-4-yl)-4-(carboxypropanamido)-2-methyl pentanoic acid impurity in an amount of less than 0.2% by weight.

Sacubitril; valsartan sodium film coated tablets were packed into blister packs of 10's count were stored for three months at 40° C./75% RH, 30° C./65% RH and 25° C./60% RH using silica gel and molecular sieves to ensure the stability conditions.

In yet another aspect of the present invention relates to use of present composition to reduce the risk of cardiovascular death and hospitalization for heart failure in patients with chronic heart failure (NYHA Class II-IV) and reduced ejection fraction.

### EXAMPLES

The following examples further illustrate the invention and do not limit the scope of the invention.

### Example 1

### Pharmaceutical Tablet Composition of Sacubitril; Valsartan Sodium

| Ingredients | mg/tab |
|---|---|
| Amorphous sacubitril; valsartan sodium on colloidal | 377.00 |
| silicon dioxide | |
| Microcrystalline cellulose | 20.00 |
| Low substituted hydroxypropyl cellulose | 20.00 |
| Povidone | 15.00 |
| Isopropyl alcohol | q.s. |
| Crospovidone | 5.00 |
| Colloidal silicon dioxide | 1.00 |
| Magnesium stearate | 2.00 |
| Core tablet weight | 440.00 |
| Film coating: | |
| Opadry ® white 06A580019* | 18.00 |
| Isopropyl alcohol | q.s. |
| Dichloro methane | q.s. |
| Film coated tablet weight | 458.00 |

*Opadry ® white composition: hypromellose, di-acetylated monoglyceride and titanium dioxide.

Brief Manufacturing Method:

The process for the preparation of a tablet comprising amorphous sacubitril; valsartan sodium processed at a temperature of 25° C. and relative humidity of less than 50% and involves following steps:

(i) Sacubitril; valsartan sodium, microcrystalline cellulose and low substituted hydroxypropyl cellulose were sifted and blended,

(ii) non-aqueous granulating solution was prepared by dissolving povidone in isopropyl alcohol,

(iii) blend of step (i) was granulated using non-aqueous solution of step (ii), followed by, drying and milling to get the desired granules,

(iv) crospovidone and colloidal silicon dioxide were sifted,

(v) granules of step (iii) were blended with sifted materials of step (iv),

(vi) magnesium stearate was sifted,

(vii) blend of step (v) was lubricated with sifted magnesium stearate of step (vi),

(viii) lubricated blend of step (vii) was compressed into tablets, and finally,

(ix) tablets of the step (viii) were film coated using non-aqueous based Opadry® white 06A580019 film coating dispersion.

### Example 2

### Pharmaceutical Tablet Composition of Sacubitril; Valsartan Sodium

| Ingredients | mg/tab |
|---|---|
| Amorphous sacubitril; valsartan sodium on colloidal | 350.00 |
| silicon dioxide | |
| Microcrystalline cellulose | 64.00 |
| Low substituted hydroxypropyl cellulose | 8.00 |
| Povidone | 12.50 |
| Isopropyl alcohol | q.s. |
| Crospovidone | 2.50 |
| Colloidal silicon dioxide | 1.00 |
| Magnesium stearate | 2.00 |
| Core tablet weight | 440.00 |
| Film coating: | |
| Opadry ® white 06A580019* | 18.00 |
| Isopropyl alcohol | q.s. |
| Dichloro methane | q.s. |
| Film coated tablet weight | 458.00 |

Dissolution Study:

| | |
|---|---|
| Dissolution medium | pH 6.8 Phosphate Buffer |
| Volume | 900 ml |
| Apparatus | USP II |
| Speed | 50 RPM |

| | Example 2 | |
|---|---|---|
| Time in minutes | % of Sacubitril dissolved | % of Valsartan dissolved |
| 5 | 17 | 17 |
| 10 | 51 | 52 |
| 15 | 72 | 75 |
| 20 | 85 | 88 |
| 30 | 92 | 95 |
| 45 | 95 | 98 |

Stability Study:

Sacubitril; valsartan sodium blister packs of 10's count were stored for three months at 40° C./75% RH and 25° C./60% RH, results are as follows:

Amorphous Form Stability:

| Month | 40° C./75% RH | 25° C./60% RH |
|---|---|---|
| Initial | Intact in amorphous form | Intact in amorphous form |
| 3 months | Intact in amorphous form | Intact in amorphous form |

Above data reveals that amorphous sacubitril; valsartan sodium is intact up to three months in tablets.

Impurity Profile:

| | 40° C./75% RH | | 25° C./60% RH | |
|---|---|---|---|---|
| Impurity | Initial | 3 months | Initial | 3 months |
| (2R, 4S)-5-([1,1′-biphenyl]-4-yl)- | 0.07 | 0.10 | 0.07 | 0.08 |
| 4-(carboxypropanamido)-2- | | | | |
| methyl pentanoic acid impurity | | | | |

Above data reveals that (2R, 4S)-5-([1,1′-biphenyl]-4-yl)-4-(carboxypropanamido)-2-methyl pentanoic acid impurity is within the acceptable limits i.e. less than 0.2% by weight.

### Example 3

The composition of Example 2 was prepared at a temperature of 25° C. and relative humidity of 60%, which results more than 0.2% by weight of (2R, 4S)-5-([1,1′-biphenyl]-4-yl)-4-(Carboxypropanamido)-2-methyl pentanoic acid impurity.

### Example 4

### Pharmaceutical Tablet Composition of Sacubitril; Valsartan Sodium

| Ingredients | mg/tab |
|---|---|
| Amorphous sacubitril; valsartan sodium on colloidal | 300.00 |
| silicon dioxide | |
| Microcrystalline cellulose | 20.00 |
| Hydroxypropyl cellulose | 20.00 |
| Povidone | 15.00 |
| Isopropyl alcohol | q.s. |
| Croscarmellose sodium | 5.00 |
| Colloidal silicon dioxide | 1.00 |
| Magnesium stearate | 2.00 |
| Core tablet weight | 363.00 |
| Film coating: | |
| Opadry ® white 06A580019 | 18.00 |
| Isopropyl alcohol | q.s. |
| Dichloro methane | q.s. |
| Film coated tablet weight | 381.00 |

Brief Manufacturing Method:

The process for the preparation of a tablet comprising amorphous sacubitril; valsartan sodium processed at a temperature of 25° C. and relative humidity of less than 50% and involves following steps:

(i) Sacubitril; valsartan sodium, microcrystalline cellulose and hydroxypropyl cellulose were sifted and blended,

(ii) non-aqueous granulating solution was prepared by dissolving povidone in isopropyl alcohol,

(iii) blend of step (i) was granulated using non-aqueous solution of step (ii), followed by, drying and milling to get the desired granules,

(iv) croscarmellose sodium and colloidal silicon dioxide were sifted,

(v) granules of step (iii) were blended with sifted materials of step (iv),

(vi) magnesium stearate was sifted,

(vii) blend of step (v) was lubricated with sifted magnesium stearate of step (vi),

(viii) lubricated blend of step (vii) was compressed into tablets, and finally,

(ix) tablets of the step (viii) were film coated using non-aqueous based Opadry® white 06A580019 film coating dispersion.

### Example 5

### Pharmaceutical Tablet Composition of Sacubitril; Valsartan Sodium

| Ingredients | mg/tab |
|---|---|
| Amorphous sacubitril; valsartan sodium on colloidal | 377.00 |
| silicon dioxide | |
| Lactose monohydrate | 20.00 |
| Microcrystalline cellulose | 20.00 |
| Hydroxypropyl cellulose | 15.00 |
| Isopropyl alcohol | q.s. |
| Croscarmellose sodium | 5.00 |
| Colloidal silicon dioxide | 1.00 |
| Magnesium stearate | 2.00 |
| Core tablet weight | 463.00 |
| Film coating: | |
| Opadry ® white 06A580019 | 18.00 |
| Isopropyl alcohol | q.s. |
| Dichloro methane | q.s. |
| Film coated tablet weight | 481.00 |

Brief Manufacturing Method:

The process for the preparation of a tablet comprising amorphous sacubitril; valsartan sodium processed at a temperature of 25° C. and relative humidity of less than 50% and involves following steps:

(i) Sacubitril; valsartan sodium, lactose monohydrate and microcrystalline cellulose were sifted and blended,

(ii) non-aqueous granulating solution was prepared by dissolving hydroxypropyl cellulose in isopropyl alcohol,

(iii) blend of step (i) was granulated using non-aqueous solution of step (ii), followed by, drying and milling to get the desired granules,

(iv) croscarmellose sodium and colloidal silicon dioxide were sifted,

(v) granules of step (iii) were blended with sifted materials of step (iv),

(vi) magnesium stearate was sifted,

(vii) blend of step (v) was lubricated with sifted magnesium stearate of step (vi),

(viii) lubricated blend of step (vii) was compressed into tablets, and finally,

(ix) tablets of the step (viii) were film coated using non-aqueous based Opadry® white 06A580019 film coating dispersion.

We claim:

1. A process for preparing a tablet comprising amorphous sacubitril-valsartan sodium comprises:
(a) sifting and blending amorphous sacubitril; valsartan sodium and one or more pharmaceutically acceptable excipients;
(b) preparing binder solution by dissolving binder in at least one non-aqueous solvent;
(c) granulating the blend of step (a) using binder solution of step (b), followed by drying and milling to get the desired granules;
(d) lubricating the granules obtained in step (c) and compressing the granules into tablets;
(e) preparing film coating dispersion by dissolving a film coating polymer in at least one non-aqueous solvent, and finally,
(f) coating the tablets of step (d) using dispersion of step (e), to get coated tablets.

2. The process according to claim 1, wherein the process is carried out at a temperature of 25° C. and relative humidity of less than 50%.

3. The process according to claim 1, wherein the tablet composition obtained from the process comprises (2R, 4S)-5-([1,1′-biphenyl]-4-yl)-4-(Carboxypropanamido)-2-methyl pentanoic acid impurity in an amount of less than 0.2% by weight.

4. The process according to claim 1, wherein the film coating comprises hydroxypropyl methylcellulose.

5. The Amorphous sacubitril—valsartan sodium obtained according to the process of claim 1, wherein the amorphous sacubitril—valsartan sodium and colloidal silicon dioxide in a ratio of 1:0.5 to 1:1.

6. A tablet composition comprising amorphous sacubitril-valsartan sodium, about 1% to 20% of microcrystalline cellulose, about 1% to 10% of low-substituted hydroxypropylcellulose, about 1% to 10% povidone, about 0.1% to 10% of crospovidone, about 0.1% to 2% of colloidal silicon dioxide and about 0.1% to 2% of magnesium stearate;
wherein the tablet is prepared by non-aqueous granulation process; and
wherein the process is carried out at a temperature of 25° C. and relative humidity of less than 40%.

7. The tablet composition according to claim 6, wherein the tablet is film coated.

8. The tablet composition according to claim 6, wherein the non-aqueous granulating solution comprises povidone and at least one non-aqueous solvent selected from isopropyl alcohol, dichloro methane, methanol, ethanol, ethyl acetate, ethyl lactate, acetone, methylenechloride, 1,1,1 trichloroethane, chloroform and combinations thereof.
